(19) United States
(12) Patent Application Publication
(10) Pub. No.: US 2021/0163925 A1
(43) Pub. Date: Jun. 3, 2021

(54) Polynucleotide Barcodes for Multiplexed Proteomics

(71) Applicant: Singular Genomics Systems, Inc., La Jolla, CA (US)

(72) Inventor: Eli N. Glezer, Del Mar, CA (US)

(21) Appl. No.: 17/169,352

(22) Filed: Feb. 5, 2021

Related U.S. Application Data
(63) Continuation of application No. PCT/US20/14449, filed on Jan. 21, 2020.
(60) Provisional application No. 62/795,474, filed on Jan. 22, 2019.

(51) Int. Cl. C12N 15/10

(52) U.S. Cl. CPC C12N 15/1065

(57) ABSTRACT

Provided herein are methods for enhanced specificity of multiplexed measurements. Methods provided herein include immunoassay reactions and/or measuring protein-protein interactions with direct sequencing readouts of DNA barcodes.

### CROSS-REFERENCES TO RELATED APPLICATIONS

This application claims the benefit of U.S. Provisional Application No. 62/795,474, filed Jan. 22, 2019, which is incorporated herein by reference in its entirety and for all purposes.

### BACKGROUND

Techniques that measure multiple proteins simultaneously are often limited by insufficient specificity. For example, in multiplexed sandwich immunoassays, a false signal can be produced when an incorrect detection antibody binds to a bead or an array element that is coated with a capture antibody. The type of incorrect interaction where a variety of components could bridge a wrong detection antibody to a capture antibody leads to an incorrect result. This is particularly problematic when measuring the concentration of analytes in biological samples, such as serum or plasma.

While carefully selected antibodies have a high degree of specificity, they are not infinitely specific. In fact, most antibodies will bind certain non-target proteins with low affinity. In practice, this limits the degree of multiplexing that can be achieved while avoiding non-specific interactions. This is one reason why most currently available commercial multiplexed assay panels are limited to 10-20 analytes, or fewer.

### SUMMARY

In view of the foregoing, there is a need for improved methods of multiplex protein detection/measurement. The present disclosure addresses this need, and provides additional benefits as well.

In an aspect, provided herein is a complex including a substrate, a first protein-binding moiety, a substrate polynucleotide barcode, a second protein-binding moiety, a protein polynucleotide barcode, and a complementary strand hybridized to one or more of the substrate polynucleotide barcode or the protein polynucleotide barcode. In embodiments, the first protein-binding moiety is attached to the substrate. The substrate polynucleotide barcode may be attached to the substrate or to the first protein-binding moiety. The first and second protein-binding moieties may be bound to each other or to an analyte. In embodiments, the protein polynucleotide barcode is attached to the second protein-binding moiety. In embodiments, the complementary strand includes a labeled nucleotide.

In an aspect, provided herein are methods of identifying a protein interaction. In embodiments, the methods include contacting a plurality of substrates with a plurality of different second protein-binding moieties where each substrate of the plurality of substrates is attached to a different first protein-binding moiety, each of the different first protein-binding moieties is a different first protein, each substrate or each different first protein-binding moiety is attached to a different substrate polynucleotide barcode, each substrate polynucleotide barcode uniquely identifies each of the different first protein-binding moieties, and each of the plurality of different second protein binding-moieties is a different second protein attached to a label, thereby forming a complex comprising one of the substrates attached to one of the different first proteins, one of the substrate polynucleotide barcodes, and one of the different second proteins attached to the label. In embodiments, the methods include synthesizing and detecting a complementary strand of the substrate polynucleotide barcode within the complex, thereby identifying an interaction between the different first protein and the different second protein within the complex.

In an aspect, provided herein are methods of detecting an analyte in a sample. In embodiments, the methods include contacting a plurality of substrates with the sample and a plurality of different second protein-binding moieties, where each substrate of the plurality of substrates is attached to a different first protein-binding moiety, each substrate or each different first protein-binding moiety is attached to a different substrate polynucleotide barcode, each substrate polynucleotide barcode uniquely identifies each of the different first protein-binding moieties, and each of the plurality of different second protein-binding moieties is attached to a label, thereby forming a complex comprising one of the substrates attached to one of the different first protein-binding moieties, one of the substrate polynucleotide barcodes, the analyte, and one of the second protein-binding moieties attached to the label. In embodiments, the method includes synthesizing and detecting a complementary strand of the substrate polynucleotide barcode within the complex, thereby detecting the analyte.

### BRIEF DESCRIPTION OF THE DRAWINGS

FIG. 1 illustrates the correct (specific) signal and incorrect (non-specific) signal interactions in a typical sandwich immunoassay. Panel (a) shows a correct specific signal interaction in which the capture antibody (c-Ab A) is specific to and binds analyte “A” and the detection antibody (d-Ab A) is specific for and binds analyte “A”. Panel (b) shows an incorrect (non-specific) signal interaction in which a capture antibody (c-Ab A) specific for analyte “A” incorrectly binds analyte “B” and the detection antibody (d-Ab B) is specific for and correctly binds analyte “B”. Panel (c) shows an incorrect signal interaction in which capture antibody (c-Ab A) and detection antibody (d-Ab B) both incorrectly bind a non-specific analyte “X”. Panel (d) shows an incorrect signal interaction in which capture antibody and detection antibody bind each other non-specifically. While interactions depicted in (b) and (d) can be evaluated in an artificial matrix, interaction as shown in panel (c) is specific to individual samples, and thus is a particularly insidious type of false signal as it is difficult to resolve and distinguish.

FIG. 2 shows an example of a bead coated with capture antibodies specific to analyte A (c-Ab A). The bead also carries a DNA barcode that identifies it as capture Bead A (DNA barcode c-A). The DNA barcode may be affixed to the bead using known methods in the art (e.g., NHS-ester coupling to an amine functionalized bead; or methods described in Kohara, Y., et al. (2002); Nucleic acids research, 30(16), e87, which is incorporated by reference). The detection antibodies carry their own unique DNA barcodes, labeled as DNA Barcode d-A or DNA Barcode d-B in the illustration. The illustration shows both a specific binding reaction (c-Ab A with d-Ab A), and a non-specific binding reaction (c-Ab A with d-Ab B).

FIG. 3 shows a set of beads at the bottom of a well plate. Commonly used well plates have 96 or 384 wells. Other well-plate formats, such as 24, 48, 1536, and 9600 wells, etc. could also be used. Bead ‘A’ contains DNA Barcode c-A and c-Ab A; similarly bead ‘B’ contains DNA Barcode c-B and c-Ab B, adopting the similar nomenclature described in FIG. 2.

FIG. 4 illustrates an example of protein-protein interactions measured with DNA barcodes. Protein A is coated onto a capture bead, for example by using biotinylated proteins on a streptavidin-coated bead. The bead also carries a DNA barcode (capture-A). Similarly, other beads are coated with other proteins and corresponding barcodes. A protein interaction is detected by reading the bead barcode (i.e., sequencing the barcode) and the barcodes of a bound protein (shown as Protein B with DNA barcode d-B).

FIG. 5 illustrates a multiplexed serology application of the methods described herein. Beads are coated with proteins or other antigens, as well as DNA barcodes that identify the beads.

### DETAILED DESCRIPTION

The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, N.Y. 1994); and Sambrook and Green, Molecular Cloning: A Laboratory Manual, 4th Edition (2012). Methods, devices and materials similar or equivalent to those described herein can be used in the practice of this invention.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term “antibody” also includes chimeric antibodies, humanized antibodies, as well as fully humanized antibodies.

As used herein, the phrase “specifically (or selectively) binds” to an antibody or “specifically (or selectively) immunoreactive with,” when referring to a protein or other analyte, refers to a binding reaction that is determinative of the presence of an analyte (e.g., a protein), often in a heterogeneous population of proteins and other biologics. Thus, under designated immunoassay conditions, the specified antibodies bind to a particular analyte at least two times the background and more typically more than 10 to 100 times background. Specific binding to an antibody under such conditions utilizes an antibody that is selected for its specificity for a particular analyte. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular analyte. In embodiments, specific binding entails a binding affinity, expressed as a K_{D} (such as a K_{D} measured by surface plasmon resonance at an appropriate temperature, such as 37° C.). In embodiments, the K_{D} of a specific binding interaction is less than about 100 nM, 50 nM, 10 nM, 1 nM, 0.05 nM, or lower. In embodiments, the K_{D} of a specific binding interaction is about 0.01-100 nM, 0.1-50 nM, or 1-10 nM. In embodiments, the K_{D} of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically a specific or selective reaction will be at least twice background signal to noise and more typically more than 10 to 100 times greater than background.

An example immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) 1 Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid surface. An association may refer to covalent or non-covalent means for attaching labels to solid or semi-solid supports such as beads. An association may comprise hybridization between a target and a label.

As used herein, the term “barcode” refers to a known nucleic acid sequence that allows some feature with which the barcode is associated to be identified. Typically, a barcode is unique to a particular feature in a pool of barcodes that differ from one another in sequence, and each of which is associated with a different feature. For example, each of a plurality of proteins in a pool of proteins may be uniquely associated with a different barcode sequence, such that isolating or localizing one of the proteins and sequencing the barcode identifies the protein that was isolated or localized. When a barcode is associated with a particular binding moiety, it may be attached directly (e.g., bound or cross-linked to the binding moiety) or indirectly (e.g., bound to a substrate, such as a bead, that is also bound to the binding moiety). Moreover, when a barcode is associated with a binding moiety that specifically binds a particular analyte, sequencing the barcode may also be used to detect the presence of the analyte. In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are 10-50 nucleotides in length, such as 15-40 or 20-30 nucleotides in length. In a pool of different barcodes, barcodes may have the same or different lengths. In general, barcodes are of sufficient length and comprise sequences that are sufficiently different to allow the identification of associated features (e.g., a binding moiety or analyte) based on barcodes with which they are associated. In embodiments, a barcode can be identified accurately after the mutation, insertion, or deletion of one or more nucleotides in the barcode sequence, such as the mutation, insertion, or deletion of 1, 2, 3, 4, 5, or more nucleotides. In embodiments, each barcode in a plurality of barcodes differs from every other barcode in the plurality by at least three nucleotide positions, such as at least 3, 4, 5, 6, 7, 8, 9, 10, or more nucleotide positions.

As used herein, a “biological sample” encompasses essentially any sample type obtained from a subject that can be used in the methods described herein. The biological sample may be any bodily fluid, tissue or any other suitable sample. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as cells, polypeptides, or proteins. The term “biological sample” encompasses a clinical sample, but also, in some instances, includes cells in culture, cell supernatants, cell lysates, blood, serum, plasma, urine, cerebral spinal fluid, biological fluid, and tissue samples.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, a protein or enzyme.

As used herein, the term “expression” includes any step involved in the production of the polypeptide including, but not limited to, transcription, post-transcriptional modification, translation, post-translational modification, and secretion.

As used herein, the term “immunoassay” refers to a biochemical test that measures the presence or concentration of a macromolecule or a small molecule in a solution involving a reaction between an antibody and an antigen. The molecule detected by the immunoassay is often referred to as an “analyte” and is in many cases a protein, although it may be other kinds of molecules, of different sizes and types. Immunoassays come in many different formats and variations. Immunoassays may be run in multiple steps with reagents being added and washed away or separated at different points in the assay. Multi-step assays are often called separation immunoassays or heterogeneous immunoassays. Some immunoassays can be carried out simply by mixing the reagents and sample and making a physical measurement. Such assays are called homogenous immunoassays or less frequently non-separation immunoassays. Immunoassays include assays in which the analyte is an antigen, as well as assays in which the analyte is an antibody (e.g., when detecting the presence, absence, or degree of an immune response). In embodiments, an immunoassay includes detecting multiple different analytes from a single sample simultaneously in a common reaction volume.

As used herein, the term “nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

As used herein, the term “polynucleotide primer” refers to any polynucleotide molecule that may hybridize to a polynucleotide template, be bound by a polymerase, and be extended in a template-directed process for nucleic acid synthesis. The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin).

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “analogue”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide useful in practicing the invention, a nucleotide analog refers to a compound that, like the nucleotide of which it is an analog, can be incorporated into a nucleic acid molecule (e.g., an extension product) by a suitable polymerase, for example, a DNA polymerase in the context of a dNTP analogue. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both.

As used herein, the terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. In various embodiments, detecting the concentrations of analytes (e.g., marker proteins) in a biological sample is contemplated for use within diagnostic, prognostic, or monitoring methods disclosed herein. The term also includes fusion proteins, including, but not limited to, naturally occurring fusion proteins with a heterologous amino acid sequence, fusions with heterologous and homologous leader sequences, with or without N-terminal methionine residues; immunologically tagged proteins; and the like. The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

For specific proteins described herein, the named protein includes any of the protein's naturally occurring forms, variants or homologs that maintain the protein activity, such as a protein's ability to specifically bind a particular target (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form.

As used herein, the term “proteomics” refers to the large-scale experimental analysis of proteins and proteomes (e.g., the entire set of proteins that are produced or modified by an organism or system, or a subset thereof under particular conditions). It is more complicated than genomics because an organism's genome is more or less constant, whereas the proteome differs from cell to cell and from time to time. Distinct genes are expressed in different cell types, which means that even the basic set of proteins that are produced in a cell needs to be identified. This phenomenon has been studied by RNA analysis, but it was found not to correlate with protein content. mRNA is not always translated into protein, and the amount of protein produced for a given amount of mRNA depends on the gene it is transcribed from and on the current physiological state of the cell. Proteomics confirms the presence of the protein and provides a direct measure of the quantity present.

As used herein, the term “label” or “labels” generally refer to molecules that can directly or indirectly produce or result in a detectable signal either by themselves or upon interaction with another molecule. Non-limiting examples of detectable labels include labels comprising fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.).

As used herein, the term “solid support” refers to discrete solid or semi-solid surfaces to which a plurality of barcodes and/or binding moieties (e.g., proteins) may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A solid support may be used interchangeably with the term “bead.”

As used herein, the term “selective” or “selectivity” or the like of a compound refers to the compound's ability to discriminate between molecular targets.

As used herein, the terms “specific”, “specifically”, “specificity”, or the like of a compound refers to the compound's ability to cause a particular action, such as binding, to a particular molecular target with minimal or no action to other proteins in the cell.

The terms “bind” and “bound” as used herein are used in accordance with thier plain and ordinary meanings and refer to an association between atoms or molecules. The association can be direct or indirect. For example, bound atoms or molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules (e.g., as in a substrate, bound to a first antibody, bound to an analyte, bound to a second antibody), thereby forming a complex.

As used herein, the terms “protein binding moiety” or “protein-binding moiety” refers to a compound or molecule, or portion thereof, capable of binding or interacting with a protein. In embodiments, a protein binding moiety specifically binds a particular protein (e.g., a protein antigen or epitope thereof). In embodiments a protein binding moiety is an immunoglobulin (IgA, IgD, IgE, IgG, or IgM). Intact immunoglobulins, also known as antibodies, are typically tetrameric glycosylated proteins composed of two light (L) chains of approximately 25 kDa each, and two heavy (H) chains of approximately 50 kDa each. In embodiments, the protein binding moiety is an antigen-specific antibody. Non-limiting examples of protein-binding moieties encompassed within the term “antigen-specific antibody” used herein include: (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; and (vi) an isolated CDR. Furthermore, although the two domains of the Fv fragment, VL and VH, are coded for by separate genes, they may be recombinantly joined by a synthetic linker, creating a single protein chain in which the VL and VH domains pair to form monovalent molecules (known as single chain Fv (scFv)). The most commonly used linker is a 15-residue (Gly4Ser)3 peptide, but other linkers are also known in the art. Single chain antibodies are also intended to be encompassed within the terms “protein-binding moiety,” of an antibody. The antibody can also be a polyclonal antibody, monoclonal antibody, chimeric antibody, antigen-binding fragment, Fc fragment, single chain antibodies, or any derivatives thereof. In embodiments, the protein-binding moiety is the antigen-binding site (e.g., fragment antigen-binding (Fab) variable region) of an antibody. The term “antigen-binding site” of an antibody (or simply “antibody portion”), as used herein, refers to one or more fragments of an antibody that retains the ability to specifically bind to an antigen. It has been shown that the antigen-binding function of an antibody can be performed by fragments of a full-length antibody.

As used herein, the term “analyte” refers to a component, substance, or constituent of interest in an analytical procedure whose presence, absence, or amount is desired to be determined or measured. In an immunoassay, for example, the analyte may be a protein, protein fragment, polypeptide, an antibody, or a molecule detectable with an antibody.

As used herein, the term “substrate” refers to a discrete solid or semi-solid surface to which a plurality of protein binding moieties, polynucleotide barcodes, and/or complementary polynucleotide strands may be attached. A substrate may encompass any type of solid, porous, or hollow sphere, ball, bearing, cylinder, or other similar configuration composed of plastic, ceramic, metal, or polymeric material (e.g., hydrogel) onto which a nucleic acid or protein including protein fragment may be immobilized (e.g., covalently or non-covalently). A substrate may include a discrete particle that may be spherical (e.g., microspheres) or have a non-spherical or irregular shape, such as cubic, cuboid, pyramidal, cylindrical, conical, oblong, or disc-shaped, and the like. In embodiments, the substrate is a bead. A bead can be spherical or non-spherical in shape.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Complex

In an aspect, provided herein is a complex including a substrate, a first protein-binding moiety, a substrate polynucleotide barcode, a second protein-binding moiety, a protein polynucleotide barcode, and a complementary strand hybridized to one or more of the substrate polynucleotide barcode or the protein polynucleotide barcode. In embodiments, the first protein-binding moiety is attached to the substrate. The substrate polynucleotide barcode may be attached to the substrate or to the first protein-binding moiety. The first and second protein-binding moieties may be bound to each other or to an analyte. In embodiments, the protein polynucleotide barcode is attached to the second protein-binding moiety. In embodiments, the complementary strand includes a labeled nucleotide. In embodiments, the protein-binding moiety is an antibody. In embodiments, the first protein-binding moiety and the second protein-binding moiety bind the same protein.

In embodiments, the substrate is a bead, a magnetic bead, a paramagnetic bead, or a hydrogel particle. In embodiments, the substrate is a bead. In embodiments, the substrate is a magnetic bead. In embodiments, the substrate is a paramagnetic bead. In embodiments, the substrate is a hydrogel particle. Examples of hydrogels include, but are not limited to agarose- and acrylamide-based gels, such as polyacrylamide, poly-N-isopropylacrylamide, poly N-isopropylpolyacrylamide, 2-hydroxyethyl acrylate and methacrylate, zwitterionic monomers, polyethylene glycol acrylate and methacrylate. Beads may be made of any of a variety of suitable materials, and take any of a variety of shapes (which may be substantially uniform or non-uniform across a plurality of beads in a composition). Beads may be solid, semi-solid, or porous. Beads may be spherical (e.g., microspheres) or have a non-spherical or irregular shape, such as cubic, cuboid, pyramidal, cylindrical, conical, oblong, or disc-shaped, and the like. Beads may be composed of a plastic, ceramic, metal, or polymeric material (e.g., hydrogel) onto which a nucleic acid may be immobilized (e.g., covalently or non-covalently). In embodiments, the beads are core-shell particles, that is a solid, semi-solid, or porous core that is surrounded by a shell polymer in the sense that the shell polymer completely covers each core, and no core is in direct contact with any other core. In embodiments, the shell polymer has a higher permeability than the core polymer. In embodiments, the core contains the first protein-binding moiety. In embodiments, the shell contains the first protein-binding moiety. In embodiments, the bead is a plastic bead with a magnetic core (e.g., micro magnetic beads coated in a polymer such as Dynabeads), a glass bead, or a glass bead with a magnetic core. In embodiments, the bead is further coated in a polymer which would then collectively serve as the substrate (i.e., the polymber contains the first protein-binding moiety). In embodiments, the bead further includes an inert (i.e., non-reactive) polymeric shell. The inert polymeric shell is optionally permeable to analytes, solvents, and proteins. Core polymers and shell polymers can comprise any of a variety of polymers. In embodiments, the substrate includes a solid bead support (which itself may include a magnetic core and an encapsulating polymer layer), a polymeric core layer around the bead for containing the first protein-binding moiety, and a shell polymer layer.

In embodiments, the first protein-binding moiety is attached to the substrate. Attachment can be covalent or non-covalent. In embodiments, the attachment is a covalent bond. In embodiments, the first protein-binding moiety is selected from a protein, an aptamer, an antibody, an antigen-binding antibody fragment, an antigen, a receptor, and a ligand. In an embodiment, the first protein-binding moiety is a protein. In an embodiment, the first protein-binding moiety is an aptamer. In an embodiment, the first protein-binding moiety is an antibody. In an embodiment, the first protein-binding moiety is an antigen-binding antibody fragment. In an embodiment, the first protein-binding moiety is an antigen. In an embodiment, the first protein-binding moiety is a receptor. In an embodiment, the first protein-binding moiety is a ligand. In embodiments, compositions comprise a plurality of different complexes, where each of the different complexes is characterized by a different first protein-binding moiety. The plurality of first protein-binding moieties may be of the same or different types. For example, all of the first protein-binding moieties may be antibodies, or antigen-binding fragments thereof. As a further example, the plurality of first protein-binding moieties may include a combination of one or more (or all) of proteins, aptamers, antibodies, antigens, receptors, and ligands. In embodiments, the first protein-binding moiety is IgA, IgD, IgE, IgG, or IgM. In embodiments, the first protein-binding moiety is IgE.

In embodiments, the second protein-binding moiety is selected from a protein, an aptamer, an antibody, an antigen-binding antibody fragment, an antigen, a receptor, and a ligand. In an embodiment, the second protein-binding moiety is a protein. In an embodiment, the second protein-binding moiety is an aptamer. In an embodiment, the second protein-binding moiety is an antibody. In an embodiment, the second protein-binding moiety is an antigen-binding antibody fragment. In an embodiment, the second protein-binding moiety is an antigen. In an embodiment, the second protein-binding moiety is a receptor. In an embodiment, the second protein-binding moiety is a ligand. In embodiments, compositions comprise a plurality of different complexes, where each of the different complexes is characterized by a different second protein-binding moiety. The plurality of second protein-binding moieties may be of the same or different types. For example, all of the second protein-binding moieties may be antibodies, or antigen-binding fragments thereof. As a further example, the plurality of second protein-binding moieties may include a combination of one or more (or all) of proteins, aptamers, antibodies, antigens, receptors, and ligands. In embodiments, the second protein-binding moiety is IgA, IgD, IgE, IgG, or IgM. In embodiments, the second protein-binding moiety is IgE.

First and second protein-binding moieties bound directly or indirectly in a complex may be of the same or different types. In embodiments, the first protein-binding moiety is a first protein or an aptamer and the second protein-binding moiety is a second protein or an aptamer. In embodiments, the first protein-binding moiety is a first protein and the second protein-binding moiety is a second protein. In embodiments, the first protein-binding moiety is a first protein and the second protein-binding moiety is an aptamer. In embodiments, the first protein-binding moiety is an aptamer and the second protein-binding moiety is a second protein. In embodiments, the first protein-binding moiety is an aptamer and the second protein-binding moiety is an aptamer. In embodiments, both the first protein and second protein are antibodies, or antigen binding fragments thereof.

In embodiments, the second protein-binding moiety is attached to a first-protein binding moiety, directly or indirectly. In embodiments, the first protein-binding moiety is bound directly to the second protein-binding moiety, such as in a protein-protein interaction (which may optionally be stabilized by cross-linking). In embodiments, the first protein-binding moiety and the second protein-binding moiety are non-covalently bound to an analyte, thereby forming a complex between the first protein-binding moiety, the second protein-binding moiety, and the analyte (which may optionally be stabilized by cross-linking).

In embodiments, the first protein-binding moiety and the second protein-binding moiety are cross-linked. Crosslinking is the process of joining two or more molecules, such as by a covalent bond, non-covalent interactions, or interactions with one or more intermediate molecules. Examples of crosslinking reagents (or crosslinkers) include molecules that contain two or more reactive ends capable of chemically attaching to specific functional groups (e.g., primary amines, sulfhydryls, etc.) on proteins or other molecules. The crosslinking may be direct, for example, a covalent bond. The cross-linking may be indirect, for example, a complex of antibodies that join the first protein-binding moiety to the second protein-binding moiety (which may optionally be stabilized by further cross-linking). By way of example, the complex of antibodies may include anti-mouse antibody binding moieties and/or Fc antibodies. Various cross-linking reagents and processes are available, particularly for cross-linking proteins. Examples of some common crosslinkers are the imidoester crosslinker dimethyl suberimidate, the N-Hydroxysuccinimide-ester crosslinker BS3 and formaldehyde. Each of these crosslinkers induces nucleophilic attack of the amino group of lysine and subsequent covalent bonding via the crosslinker. The zero-length carbodiimide crosslinker EDC functions by converting carboxyls into amine-reactive isourea intermediates that bind to lysine residues or other available primary amines. SMCC or its water-soluble analog, Sulfo-SMCC, is commonly used to prepare antibody-hapten conjugates for antibody development. An in-vitro cross-linking method, termed PICUP (photo-induced cross-linking of unmodified proteins) is a process in which ammonium persulfate (APS), which acts as an electron acceptor, and tris-bipyridylruthenium (II) cation ([Ru(bpy)3]²⁺) are added to the protein of interest and irradiated with UV light. In-vivo crosslinking of protein complexes using photo-reactive amino acid analogs is a method in which cells are grown with photoreactive diazirine analogs to leucine and methionine, which are incorporated into proteins. Upon exposure to ultraviolet light, the diazirines are activated and bind to interacting proteins that are within a few angstroms of the photo-reactive amino acid analog (UV cross-linking).

In embodiments, the analyte is a protein, an antibody, an antigen, or a ligand. In embodiments, the analyte is a protein. In embodiments, the analyte is an antibody. In embodiments, the analyte is an antigen. In embodiments, the analyte is a ligand. In embodiments, the analyte is an antigen expressing antibody. In embodiments, the analyte is a molecule (e.g., organic or inorganic molecule). In embodiments, the analyte is a peptide, protein, or glycoprotein. In embodiments, the analyte is an amino acid, carbohydrate, nucleic acid, lipid, or toxin. In embodiments, the analyte is a steroid. In embodiments, the analyte is a vitamin. In embodiments, the analyte is a virus or virus particles. Analytes to be detected also include, but are not limited to, neurotransmitters, hormones, growth factors, antineoplastic agents, cytokines, monokines, lymphokines, nutrients, enzymes, receptors, antibacterial agents, antiviral agents and antifungal agents, and combinations thereof. The term “analyte” also refers to detectable components of structured elements such as cells, including all animal and plant cells, and microorganisms, such as fungi, viruses, bacteria including, but not limited to, all gram positive and gram negative bacteria, and protozoa. In embodiments, the analyte will have at least one epitope that an antibody or a protein-binding moiety can recognize.

In embodiments, the protein-binding moiety is a protein. In embodiments the analyte is a protein. The protein may be a full length protein or protein fragment. The protein may be an antibody or antigen-binding antibody fragment. The protein may be a hormone, cytokine, glycoprotein such as an interleukin, growth factor, or a receptor. The protein may be native to a subject being tested (e.g. a human), foreign to a subject being tested (e.g. viral or bacterial), mutant versions of proteins normally found in a subject being tested, or a library of proteins of a particular type (e.g., the proteome of a particular subject, tissue, or cell).

In embodiments, the protein-binding moiety is an aptamer. In general, aptamers are oligonucleotide or peptide molecules that bind to a specific target molecule. Aptamers may be created by selecting them from a large random sequence pool. Aptamers can be classified as DNA or RNA or XNA (nucleic acid analogue) aptamers. In embodiments, aptamers consist of (usually short) strands of oligonucleotides. In embodiments, peptide aptamers consist of one (or more) short variable peptide domains, attached at both ends to a protein scaffold. Nucleic acid aptamers are nucleic acid species that are typically the product of engineering through repeated rounds of in vitro selection, such as SELEX (systematic evolution of ligands by exponential enrichment), to bind to various molecular targets such as small molecules, proteins, nucleic acids, and even cells, tissues and organisms. At the molecular level, aptamers bind to its target site through non-covalent interactions. Aptamers bind to these specific targets because of electrostatic interactions, hydrophobic interactions, and their complementary shapes. In embodiments, peptide aptamers are artificial proteins selected or engineered to bind specific target molecules. These proteins may include or consist of one or more peptide loops of variable sequence displayed by a protein scaffold. They are typically isolated from combinatorial libraries and often subsequently improved by directed mutation or rounds of variable region mutagenesis and selection.

In embodiments, the protein-binding moiety is an antigen. In general, antigens include molecules or portions thereof that trigger an immune response in a host (e.g., in a subject), and may be recognized by an antibody. Antigens may be foreign to a subject (e.g., as in viral or bacterial proteins, polysaccharides, or other molecules), or native to the subject (e.g., as in an autoimmune response to self-proteins, which optionally may be mutant forms of a native protein). Examples of antigens include, without limitation, viral antigens, bacterial antigens, fungal antigens, cancer or tumor antigens, and allergens. Examples of viral antigens include, but are not limited to, env, gag, rev, tar, tat, nucleocapsid proteins and reverse transcriptase from immunodeficiency viruses (e.g., HIV, FIV), such as HIV-1 gag, HIV-1 env, HIV-1 pol, HIV-1 tat, HIV-1 nef; HBV surface antigen and core antigen, HbsAG, HbcAg; HCV antigens such as hepatitis C core antigen; influenza nucleocapsid proteins; parainfluenza nucleocapsid proteins; HPV E6 and E7 such as human papilloma type 16 E6 and E7 proteins; Epstein-Barr virus LMP-1, LMP-2 and EBNA-2; herpes LAA and glycoprotein D such as HSV glycoprotein D; as well as similar proteins from other viruses. In embodiments, the protein-binding moiety is an antibody that is reactive to a plurality of viral antigens within the same viral group. For example, a flavivirus group-reactive antibody such as the monoclonal antibody MAb 6B6C-1, dengue 4G2, or Murray Valley 4A1B-9 is reactive with arbovirus antigens within the flavivirus genus, which includes the West Nile virus, Saint Louis encephalitis virus, Japanese encephalitis virus, and dengue virus. Similarly, for example, an alphavirus group-reactive antibody such as EEE 1A4B-6 or WEE 2A2C-3 is reactive with alphavirus antigens within the alphavirus genus, which includes eastern equine encephalitis virus, western equine encephalitis virus, and Venezuelan equine encephalitis virus. Similarly, for example, a bunyavirus group-reactive antibody such as LAC 10G5.4 is reactive with bunyavirus antigens within the bunyavirus genus, which includes the California serogroup of bunyaviruses, which includes La Crosse virus. Examples of bacterial antigens include, but are not limited, to capsule antigens (e.g., protein or polysaccharide antigens such as CP5 or CP8 from the S. aureus capsule); cell wall (including outer membrane) antigens such as peptidoglycan (e.g., mucopeptides, glycopeptides, mureins, muramic acid residues, and glucose amine residues) polysaccharides, teichoic acids (e.g., ribitol teichoic acids and glycerol teichoic acids), phospholipids, hopanoids, and lipopolysaccharides (e.g., the lipid A or O-polysaccharide moieties of bacteria such as Pseudomonas aeruginosa serotype O11); plasma membrane components including phospholipids, hopanoids, and proteins; proteins and peptidoglycan found within the periplasm; fimbrae antigens, pili antigens, flagellar antigens, and S-layer antigens. S. aureus antigens can be a serotype 5 capsular antigen, a serotype 8 capsular antigen, and antigen shared by serotypes 5 and 8 capsular antigens, a serotype 336 capsular antigen, protein A, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, Sbi, and von Willebrand factor binding protein. Non-limiting examples of fungal antigens include, but are not limited to, Candida fungal antigen components; Histoplasma fungal antigens such as heat shock protein 60 (HSP60) and other Histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. Examples of cancer antigens include, but are not limited to, MAGE, MART-1/Melan-A, gplOO, dipeptidyl peptidase IV (DPPIV), adenosine deaminase-binding protein (ADAbp), cyclophilin b, colorectal associated antigen (CRC)-COI 7-1 A/GA733, carcinoembryonic antigen (CEA) and its immunogenic epitopes CAP-1 and CAP-2, etvβ, aml1, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, Smad family of tumor antigens, lmp-1, P1 A, EBV-encoded nuclear antigen (EBNA)-I, brain glycogen phosphorylase, SSX-1, SSX-2 (HOM-MEL-40), SSX-1, SSX-4, SSX-5, SCP-1, CT-7, and c-erbB-2. Examples of allergens include, but are not limited to, dust, pollen, pet dander, food such as peanuts, nuts, shellfish, fish, wheat milk, eggs, soy and their derivatives, and sulphgites. These lists are not meant to be limiting.

In embodiments, the protein-binding moiety is a receptor or a ligand-binding portion thereof. In general, receptors include proteins that transmit a signal in a signaling pathway in response to binding a ligand. Receptors may be intracellular receptors or cell surface receptors. Examples of cell surface receptors include ligand-gated ion channels, G protein-coupled receptors, and receptor tyrosine kinates. Examples of receptors include, without limitation, tyrosine kinase receptor, such as a colony stimulating factor 1 (CSF-1), platelet-derived growth factor (PDGF), epidermal growth factor (EGF), transforming growth factor (TGF), nerve growth factor (NGF), insulin, insulin-like growth factor 1 (IGF-1) receptor, etc.; a G-protein coupled receptor, such as a Gi-coupled, Gq-coupled or Gs-coupled receptor, e.g. a muscarinic receptor (e.g. the subtypes m1, m2, m3, m4, m5), dopamine receptor (e.g. the subtypes D1, D2, D4, D5), opiate receptor (e.g. the subtypes μ or δ), adrenergic receptor (e.g. the subtypes α1A, α1B, α1C, α2C10, α2C2, α2C4), serotonin receptor, tachykinin receptor, luteinising hormone receptor or thyroid-stimulating hormone receptor, retinoic acid/steroid super family of receptors, mutant forms of receptors such as mutant trk A receptor, mutant EGF receptors, ligand-gated channels including subtypes of nicotinic acetylcholine receptors, GABA receptors, glutamate receptors (NMDA or other subtypes), subtype 3 of the serotonin receptor, and the cAMP-regulated channel.

In embodiments, a protein-binding moiety is a ligand. In general, ligands include proteins that bind to and alter the function of a protein (e.g., an enzyme or a receptor). Ligands may be other proteins, protein fragments, or other molecules. Non-limiting examples of ligands include peptides, polypeptides or proteins, such as cytokines or growth factors. For example, ligands include but are not limited to βc, Cyclophilin A, IL-1, IL-2, IL-3, IL-4, IL-5, IL-6, IL-7, IL-8, IL-10, IL-12, IL-13, G-CSF, M-CSF, GM-CSF, BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, LIF, MCP1, MCP2, KC, MCP3, MCP4, MCPS, M-CSF, MIP1, MIP2, NOF, NT 3, NT4, NTS, NT6, NT7, OSM, PBP, PBSF, PDGF, PECAM-1, PF4, RANTES, SCF, TGFα, TGFβ₁, TGFβ₂, TGFβ₃, TNFα, TNFβ, TPO, VEGF, GH, chemokines, and eotaxin (eotaxin-1, -2 or -3).

In embodiments, the complexes provided herein include barcodes. The barcodes may include one or more barcode sequences. In some embodiments, a barcode sequence can include a nucleic acid sequence, for example a polynucleotide sequence. The polynucleotide barcode sequence may provide identifying information for the specific type of species attached to the barcode. The species may be a substrate, a first protein-binding moiety, or a second protein-binding moiety. The polynucleotide barcode sequence may form part of a longer polynucleotide that includes other elements, such as a primer-binding site or a target-binding site. In embodiments, the primer binding site is a common sequence shared by a plurality of polynucleotides comprising different barcodes, such that a complementary primer can be used to hybridize to and sequence the plurality of different barcodes in a single reaction. In embodiments, a polynucleotide comprising the barcode comprises a loop region and a 3′ self-complementary region that hybridizes to a 5′ region of the polynucleotide to form a hairpin structure, the 3′ end of which can be extended by a polymerase. In cases where the barcode comprises a hairpin structure, sequencing the barcode can be performed without a separate primer. In embodiments, measuring the amount of a particular barcode sequence (e.g., a signal intensity associated with a particular barcode) provides a measure of the amount of an analyte in a sample, which measure may be normalized to a control or standard present in a known amount.

In embodiments, a barcode is a degenerate or partially-degenerate sequence, such that one or more nucleotides are selected at random from a set of two or more different nucleotides at one or more positions, with each of the different nucleotides selected at one or more positions represented in a pool of oligonucleotides comprising the degenerate or partially-degenerate sequence. The number of possible barcodes in a given set of barcodes will vary with the number of degenerate positions, and the number of bases permitted at each such position. For example, a barcode of five nucleotides (consecutive or non-consecutive), in which each position can be any of A, T, G, or C represents 5⁴, or 1024 possible barcodes. In embodiments, certain barcode sequences may be excluded from a pool, such as barcodes in which every position is the same base. In embodiments, there are about, 10², 10³ 10⁴, 10⁵, 10⁶, 10⁷, 10⁸, 10⁹, or a number or a range between any two of these values, unique nucleotide barcode sequences. In embodiments, there are at least, or at most 10², 10³ 10⁴, 10⁵, 10⁶, 10⁷, 10⁸, 10⁹ unique barcode sequences. In embodiments, a barcode is about, 1, 2, 3, 4, 5, 10, 15, 20, 25, 30, 35, 40, 45, 50, or a number or a range between any two of these values, nucleotides in length. A barcode can be at least, or at most, 1, 2, 3, 4, 5, 10, 15, 20, 25, 30, 35, 40, 45, 50, 100, or 200 nucleotides in length. In embodiments, such as where the number of barcodes exceeds the number of first and/or second protein-binding moieties, a diverse set of barcode sequences are attached to a given species. In some embodiments, a diverse set of barcode sequences are attached to a given first protein-binding moiety, or a substrate attached thereto.

In embodiments, the barcode is a substrate polynucleotide barcode, for example, a polynucleotide barcode attached to a substrate or a first protein-binding moiety. In embodiments, the barcode is a substrate polynucleotide barcode attached to a substrate. In embodiments, the barcode is a substrate polynucleotide barcode attached to a first protein-binding moiety. In embodiments, the substrate polynucleotide barcode is attached to the substrate using known techniques in the art. In embodiments, the substrate polynucleotide barcode is attached to the first protein-binding moiety using known techniques in the art.

In embodiments, the barcode is a protein polynucleotide barcode, for example, a polynucleotide barcode attached to a second protein-binding moiety. In embodiments, none of the substrate polynucleotide barcodes is the same as any of the protein polynucleotide barcodes. In other embodiments, a set of substrate polynucleotide barcodes is permitted to have barcode sequences in common with a set of protein polynucleotide barcodes. For example, both the substrate polynucleotide barcodes and the protein polynucleotide barcodes may include a random 4-mer sequence, such that one or more barcode associated with a particular first protein-binding moiety may also be associated with a particular second protein-binding moiety. In cases where a one or more barcodes are associated with both a first and second protein-binding moiety, the barcode sets may be sequenced in separate sequencing reactions (e.g., by including a different primer binding sequence for substrate polynucleotide barcodes as compared to a primer binding sequence for protein polynucleotide barcodes), so as to distinguish first protein-binding moieties from second protein-binding moieties based on the barcode sequences. For example, a sequencing process with a first primer can be used to sequence substrate polynucleotide barcodes, followed by another sequencing process with a second primer used to sequence protein polynucleotide barcodes, or vice versa.

Barcodes can be of any of a variety of lengths. Substrate polynucleotide barcodes may or may not all be the same length. Protein polynucleotide barcodes may or may not all be the same length. Substrate polynucleotide barcodes may or may not be the same length as protein polynucleotide barcodes. In embodiments, the substrate polynucleotide barcode, the protein polynucleotide barcode, or both are 1-50, 5-45, 10-40, 15-35, or 20-30 nucleotides in length. In embodiments, the substrate polynucleotide barcode, the protein polynucleotide barcode, or both are 10-50, 15-45, 20-40, or 25-35 nucleotides in length. In embodiments, the substrate polynucleotide barcode, the protein polynucleotide barcode, or both are 1-25, 2-20, 3-15, or 4-10 nucleotides in length.

Barcodes may form a portion of a longer polynucleotide that includes additional sequences or structural elements. In embodiments, complexes provided herein include a substrate polynucleotide barcode that forms a portion of a polynucleotide that is 10-100, 10-50, or 20-30 nucleotides in length; and/or a protein polynucleotide barcode that forms a portion of a polynucleotide that is 10-100, 10-50, or 20-30 nucleotides in length. In embodiments, a polynucleotide comprising a polynucleotide barcode is 10-50 nucleotides in length. In embodiments, a polynucleotide comprising a polynucleotide barcode is 20-30 nucleotides in length. Examples of additional sequences include, but are not limited to, sequences that participate in the formation of a hairpin structure (e.g., a loop sequence and a self-complementary 3′ end), and a primer binding sequence that is complementary to at least a portion of a primer (e.g., a sequencing primer). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length.

In embodiments, complexes provided herein include a complementary strand. The complementary strand includes one or more labeled nucleotides. The complementary strand may be hybridized to one or more of the substrate polynucleotide barcode or the protein polynucleotide barcode. In embodiments, the complementary strand is formed by extension of a polynucleotide primer. In embodiments, the complementary strand is formed by extension of a 3′ end of a polynucleotide that includes the polynucleotide barcode to which the complementary strand is hybridized. Priming can occur various ways in a polynucleotide extension reaction (such as in polynucleotide sequencing). In embodiments, polynucleotide extension involves extension of a separate primer polynucleotide. In embodiments, polynucleotide extension involves extending the 3′ terminus of a single-stranded oligonucleotide having a hairpin structure, which folds onto itself to initiate priming. In embodiments, the complementary strand is the product of a sequencing process.

In an aspect, the present disclosure provides compositions including one or more complexes described herein, including any of the various aspects and embodiments described herein. In embodiments, the composition includes a plurality of substrates and a plurality of different second protein-binding moieties. Each substrate of the plurality of substrates may be attached to a different first protein-binding moiety. Each substrate or each different first protein-binding moiety may be attached to a different substrate polynucleotide barcode that uniquely identifies each of the different first protein-binding moieties. Each different second protein-binding moiety of the plurality of different second protein-binding moieties may be attached to a different protein polynucleotide barcode that uniquely identifies each of the plurality of different second protein-binding moieties.

In embodiments, the plurality of substrates includes about or at least about 10, 100, 1000, 10000, 20000, 50000, or 100000 substrates or a number or a range between any two of these values. In embodiments, the plurality of substrates includes 10-100000, 100-50000, or 1000-10000 substrates. In embodiments, the plurality of substrates includes at least about 100 substrates. In embodiments, the plurality of substrates include at least about 500 substrates.

In embodiments, the plurality of different second proteins includes about or at least about 10, 100, 1000, 10000, 20000, 50000, or 100000 different second protein-binding moieties. In embodiments, the plurality of different second protein-binding moieties includes 10-100000, 100-50000, or 1000-10000 different second protein-binding moieties. In embodiments, the plurality of different second protein-binding moieties includes at least about 100 different second protein-binding moieties. In embodiments, the plurality of different second protein-binding moieties includes at least about 500 different second protein-binding moieties.

In embodiments, each of the plurality of substrates is a bead. Beads may be made of any of a variety of suitable materials, and take any of a variety of shapes (which may be substantially uniform or non-uniform across a plurality of beads in a composition). Beads may be solid, semi-solid, or porous. Beads may be spherical (e.g., microspheres) or have a non-spherical or irregular shape, such as cubic, cuboid, pyramidal, cylindrical, conical, oblong, or disc-shaped, and the like. Beads may be composed of a plastic, ceramic, metal, or polymeric material (e.g., hydrogel) onto which a nucleic acid may be immobilized (e.g., covalently or non-covalently). In embodiments, the beads are magnetic or paramagnetic.

In embodiments, the plurality of substrates are in a container. A variety of suitable containers are available. Non-limiting examples of containers include tubes of various dimensions, wells of multi-well plates (e.g., 48-well, 96-well, 192-well, and 384-well formats), channels of flow cells, and compartments of microfluidic devices. In embodiments, the container is a well of a multi-well plate.

Methods

In an aspect, provided herein are methods of identifying a protein interaction. In embodiments, the methods include contacting a plurality of substrates with a plurality of different second protein-binding moieties where each substrate of the plurality of substrates is attached to a different first protein-binding moiety, each of the different first protein-binding moieties is a different first protein, each substrate or each different first protein-binding moiety is attached to a different substrate polynucleotide barcode, each substrate polynucleotide barcode uniquely identifies each of the different first protein-binding moieties, and each of the plurality of different second protein binding-moieties is a different second protein attached to a label, thereby forming a complex comprising one of the substrates attached to one of the different first proteins, one of the substrate polynucleotide barcodes, and one of the different second proteins attached to the label. In embodiments, the methods include synthesizing and detecting a complementary strand of the substrate polynucleotide barcode within the complex, thereby identifying an interaction between the different first protein and the different second protein within the complex.

In an aspect, provided herein are methods of detecting an analyte in a sample. In embodiments, the methods include contacting a plurality of substrates with the sample and a plurality of different second protein-binding moieties, where each substrate of the plurality of substrates is attached to a different first protein-binding moiety, each substrate or each different first protein-binding moiety is attached to a different substrate polynucleotide barcode, each substrate polynucleotide barcode uniquely identifies each of the different first protein-binding moieties, and each of the plurality of different second protein-binding moieties is attached to a label, thereby forming a complex comprising one of the substrates attached to one of the different first protein-binding moieties, one of the substrate polynucleotide barcodes, the analyte, and one of the second protein-binding moieties attached to the label. In embodiments, the method includes synthesizing and detecting a complementary strand of the substrate polynucleotide barcode within the complex, thereby detecting the analyte.

In embodiments of the methods provided herein, contacting a plurality of substrates with a sample and with a plurality of different second protein-binding moieties is performed sequentially. For example, where a plurality of analytes are to be assayed, the substrates may be contacted with the sample and incubated under conditions suitable for binding between the plurality of analytes and corresponding protein-binding moieties. After allowing for the formation of complexes between the first protein-binding moieties and any analytes present in the sample, the second protein-binding moieties may be introduced (with or without intermediate washing and/or cross-linking steps). In embodiments, contacting a plurality of substrates with a sample and with a plurality of different second protein-binding moieties is performed simultaneously, such as by adding all three components into a single container and incubating under conditions suitable for forming complexes of analytes bound to both a first protein-binding moiety and a second protein-binding moiety in a single step.

In embodiments of the methods provided herein, detecting the analyte includes measuring a level of the analyte in the sample. In embodiments, measuring the level of the analyte in the sample includes forming a plurality of complexes and quantifying the label present in the complexes. The method of quantifying the label will depend on the nature of the label selected. A variety of suitable labels are available. For example, where the label is a fluorescent label, quantification may include a measure of fluorescence intensity, which may be compared to reference values or an internal control. As a further example, where the label is an enzyme that produces a detectable product (e.g., a luciferase or a peroxidase), quantification may include a measurement of the detectable product. In another example, the label is a fluorescently-tagged nucleotide, such that measurement of fluorescence intensity and wavelength in a sequencing reaction permits simultaneously determining the sequence of a barcode polynucleotide and the absolute or relative amount of that barcode, and the amount of an associated protein-binding moiety or analyte.

In embodiments, an analyte is deemed to be detected, or is measured, only when one or both of the substrate polynucleotide barcode or the protein polynucleotide barcode is associated with a protein-binding moiety that specifically binds that analyte. In embodiments, an analyte is deemed to be detected, or is measured, only when both the substrate polynucleotide barcode and the protein polynucleotide barcode are associated with a protein-binding moiety that specifically binds that analyte.

In embodiments, the analyte is any analyte disclosed herein, such as with regard to the various complexes and compositions disclosed herein. In embodiments of the methods provided herein, the analyte is selected from a protein, glycoprotein, lipid protein, protein fragment, cytokine, hormone, mutant protein, misfolded protein, antibody, antibody fragment, and ligand. In embodiments, the analyte is a protein. In embodiments, the analyte is a glycoprotein. In embodiments, the analyte is a lipid protein. In embodiments, the analyte is a protein fragment. In embodiments, the analyte is a cytokine. In embodiments, the analyte is a hormone. In embodiments, the analyte is a mutant protein. In embodiments, the analyte is a misfolded protein. In embodiments, the analyte is an antibody. In embodiments, the analyte is an antibody fragment. In embodiments, the analyte is a ligand.

In embodiments of the methods provided herein, the analyte is from a human, a bacterium, or a virus. In embodiments, the analyte is a human protein. In embodiments, the analyte is a bacterial protein. In embodiments, the protein is a pathogen protein. A pathogen protein refers to a protein that is from a pathogen. Examples of pathogens include, but are not limited to, viruses, prokaryote and pathogenic eukaryotic organisms such as unicellular pathogenic organisms and multicellular parasites. Pathogens also can include protozoan pathogens which include a stage in the life cycle where they are intracellular pathogens. Bacterial pathogens include, but are not limited to, such as bacterial pathogenic gram-positive cocci, which include but are not limited to: pneumococcal; staphylococcal; and streptococcal. Pathogenic gram-negative cocci include: meningococcal; and gonococcal. Pathogenic enteric gram-negative bacilli include: enterobacteriaceae; pseudomonas, acinetobacteria and eikenella; melioidosis; salmonella; shigellosis; hemophilus; chancroid; brucellosis; tularemia; yersinia (pasteurella); Streptobacillus moniliformis and spirilum; Listeria monocytogenes; Erysipelothrix rhusiopathiae; diphtheria; cholera; anthrax; donovanosis (granuloma inguinale); and bartonellosis. Pathogenic anaerobic bacteria include; tetanus; botulism; other clostridia; tuberculosis; leprosy; and other mycobacteria. Pathogenic spirochetal diseases include: syphilis; treponematoses: yaws, pinta and endemic syphilis; and leptospirosis. Other infections caused by higher pathogen bacteria and pathogenic fungi include: actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include rickettsial and rickettsioses. Examples of mycoplasma and chlamydial infections include: Mycoplasma pneumoniae; Lymphogranuloma venereum; psittacosis; and perinatal chlamydial infections. Pathogenic protozoans and helminths and infections eukaryotes thereby include: amebiasis; malaria; leishmaniasis; trypanosomiasis; toxoplasmosis; Pneumocystis carinii; babesiosis; giardiasis; trichinosis; filariasis; schistosomiasis; nematodes; trematodes or flukes; and cestode (tapeworm) infections. Bacteria also include E. coli, a Campylobacter, or a Salmonella. In embodiments, the analyte is a viral protein (e.g., a protein expressed by a virus). Examples of viruses include, but are not limited to, HIV, Hepatitis A, B, and C, FIV, lentiviruses, pestiviruses, West Nile Virus, measles, smallpox, cowpox, ebola, coronavirus, and the like. In embodiments, the analyte includes a cell lysate. In embodiments, the cell lysate has been clarified by centrifugation or other means to remove non-soluble materials.

In embodiments, the methods provided herein further include diagnosing a condition of a subject based on detecting the analyte. Conditions that may be diagnosed by a method disclosed herein include, without limitation, any conditions associated with the presence or absence of one or more analytes that serve as markers for the condition. Examples of conditions that may be diagnosed include, without limitation, infections, cancers, metabolic disorders, auto-immune disorders, inflammatory conditions, conditions associated with inherited mutations, and the like.

In embodiments of the methods provided herein, the first protein-binding moiety is a first protein or an aptamer and/or the second protein-binding moiety is a second protein or an aptamer. The first and second protein-binding moieties can be any of the protein-binding moieties described herein, such as with regard to the various complexes and compositions of the disclosure. In embodiments, the first protein-binding moiety is a first protein and the second protein-binding moiety is a second protein. In embodiments, the first protein-binding moiety is a first protein and the second protein-binding moiety is an aptamer. In embodiments, the first protein-binding moiety is an aptamer and the second protein-binding moiety is a second protein. In embodiments, the first protein-binding moiety is an aptamer and the second protein-binding moiety is an aptamer.

In embodiments of the methods provided herein, the label includes a protein polynucleotide barcode, each of the plurality of different second protein-binding moieties is attached to a different protein polynucleotide barcode that uniquely identifies each of the plurality of different second protein-binding moieties, and the method further comprises synthesizing and detecting a complementary strand of the protein polynucleotide barcode within the complex.

In embodiments of the methods provided herein, synthesizing and detecting a complementary strand includes polymerizing the complementary strand from labeled nucleotides, and determining the sequence of the complementary strand from the sequence of the labels of the labeled nucleotides, thereby sequencing the barcode. In embodiments of the methods provided herein, polymerizing the complementary strand includes extension of a polynucleotide primer. In embodiments, polymerizing the complementary strand includes extension of a 3′-end of a polynucleotide that includes the polynucleotide barcode to which the complementary strand is hybridized (e.g., as in a hairpin structure). In embodiments, the methods described herein further includes sequencing the barcode. The process of sequencing a barcode may employ any of a variety of suitable sequencing processes known in the art. As used herein, the term “sequencing” includes determination of partial as well as full sequence information, including the identification, ordering, or locations of the nucleotides that comprise the polynucleotide being sequenced, and inclusive of the physical processes for generating such sequence information. The term also includes the determination of the identity, ordering, and locations of one, two, or three of the four types of nucleotides within a target polynucleotide. Sequencing methods, such as those outlined in U.S. Pat. No. 5,302,509 can be carried out using the nucleotides described herein. In embodiments, sequencing comprises a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Suitable nucleotide blocking groups are also described in applications WO 2004/018497, WO 96/07669, U.S. Pat. Nos. 5,763,594, 5,808,045, 5,872,244 and 6,232,465 the contents of which are incorporated herein by reference in their entirety. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template (e.g., as in a barcode). Non-limiting examples of suitable labels are described in U.S. Pat. No. 8,178,360.

In embodiments of the methods provided herein, the plurality of different first protein-binding moieties include one or more of a protein, an antibody, an antigen-binding antibody fragment, an antigen, a receptor, a ligand, or an aptamer. In embodiments of the methods provided herein, the plurality of different first protein-binding moieties include proteins. In embodiments of the methods provided herein, the plurality of different first protein-binding moieties include antibodies. In embodiments of the methods provided herein, the plurality of different first protein-binding moieties include antigen-binding antibody fragments. In embodiments of the methods provided herein, the plurality of different first protein-binding moieties include antigens. In embodiments of the methods provided herein, the plurality of different first protein-binding moieties include receptors In embodiments of the methods provided herein, the plurality of different first protein-binding moieties include ligands. In embodiments of the methods provided herein, the plurality of different first protein-binding moieties include aptamers.

In embodiments of the methods provided herein, the plurality of different first protein-binding moieties include a plurality of human proteins, a plurality of bacterial proteins, a plurality of viral proteins, a plurality of proteins having a mutation associated with a disease or condition, or a combination thereof. In embodiments of the methods provided herein, the plurality of different first protein-binding moieties include a plurality of human proteins. In embodiments of the methods provided herein, the plurality of different first protein-binding moieties include a plurality of bacterial proteins. In embodiments of the methods provided herein, the plurality of different first protein-binding moieties include a plurality of viral proteins. In embodiments of the methods provided herein, the plurality of different first protein-binding moieties include a plurality of proteins having a mutation associated with a disease or condition.

In embodiments of the methods provided herein, the plurality of different second protein-binding moieties include one or more of a protein, an antibody, an antigen-binding antibody fragment, an antigen, a receptor, a ligand, or an aptamer. In embodiments of the methods provided herein, the plurality of different second protein-binding moieties include proteins. In embodiments of the methods provided herein, the plurality of different second protein-binding moieties include antibodies. In embodiments of the methods provided herein, the plurality of different first protein-binding moieties include antigen-binding antibody fragments. In embodiments of the methods provided herein, the plurality of different second protein-binding moieties include antigens. In embodiments of the methods provided herein, the plurality of different second protein-binding moieties include receptors. In embodiments of the methods provided herein, the plurality of different second protein-binding moieties include ligands. In embodiments of the methods provided herein, the plurality of different second protein-binding moieties include aptamers.

In embodiments of the methods provided herein, substrate polynucleotide barcodes collectively, the protein polynucleotide barcodes collectively, or both are defined by a nucleotide sequence comprising one or more degenerative or semi-degenerative sequences. In embodiments, none of the substrate polynucleotide barcodes is the same as any of the protein polynucleotide barcodes. In other embodiments, a set of substrate polynucleotide barcodes is permitted to have barcode sequences in common with a set of protein polynucleotide barcodes. The barcodes can be any of the barcodes described herein, such as with regard to the various complexes and compositions of the disclosure.

In embodiments, each of the plurality of substrates is a bead. Non-limiting examples of beads suitable for use in the present methods are provided herein, such as with regard to the various complexes and compositions of the present disclosure. In embodiments, the plurality of substrates are in a container. Non-limiting examples of containers are provided herein, such as with regard to the various complexes and compositions of the present disclosure. In embodiments, the container is a well of a multi-well plate.

In embodiments of the methods provided herein, each substrate is separated from the plurality of substrates prior to synthesizing or detecting the complementary strands. In embodiments, separating the plurality of substrates includes arranging the substrates on an array or in an emulsion. In embodiments, separating the plurality of substrates includes arranging the substrates on an array. In embodiments, separating the plurality of substrates includes arranging the substrates in an emulsion. For example, beads attached to complexes described herein may be applied to an array of wells, such that each well contains only one bead, and spaced such that signals from adjacent beads (e.g., as in a sequencing assay) can be resolved from one another. In embodiments, beads comprising complexes described herein are spread on a surface (e.g., randomly dispersed at the bottom of a well) and spaced such that signals from adjacent beads can be resolved. Spacing may be achieved physical structures on the surface (e.g., an ordered arrangement of wells), or by applying beads at a suitable density by selecting or adjusting an appropriate dilution. Optimal spacing may depend on factors such as the size of the beads, the label(s) to be detected, the field of view and/or resolution of the detector (e.g., a detector utilizing a microscope), and the like, which can be determined by those skilled in the art.

In embodiments of the methods provided herein, the plurality of substrates includes about or at least about 10, 100, 1000, 10000, 20000, 50000, or 100000 substrates or a number or a range between any two of these values, each of which is attached to a different first protein-binding moiety. In embodiments, the plurality of substrates includes 10-100000, 100-50000, or 1000-10000 substrates. In embodiments, the plurality of substrates includes at least about 100 substrates. In embodiments, the plurality of substrates include at least about 500 substrates.

In embodiments of the methods provided herein, the plurality of different second proteins includes about or at least about 10, 100, 1000, 10000, 20000, 50000, or 100000 different second protein-binding moieties. In embodiments, the plurality of different second protein-binding moieties includes 10-100000, 100-50000, or 1000-10000 different second protein-binding moieties. In embodiments, the plurality of different second protein-binding moieties includes at least about 100 different second protein-binding moieties. In embodiments, the plurality of different second protein-binding moieties includes at least about 500 different second protein-binding moieties.

In embodiments of the methods provided herein, the complementary strand of the substrate polynucleotide barcode within the complex is synthesized before or after the complementary strand of the protein polynucleotide barcode within the complex. In embodiments, the complementary strand of the substrate polynucleotide barcode within the complex is synthesized before the complementary strand of the protein polynucleotide barcode within the complex. In embodiments, the complementary strand of the substrate polynucleotide barcode within the complex is synthesized after the complementary strand of the protein polynucleotide barcode within the complex.

In embodiments, the methods provided herein further include crosslinking a first protein-binding moiety with a second protein-binding moiety (and, optionally, with an analyte). The crosslinking may be direct, for example, forming covalent bonds between two or more of the first protein-binding moiety, the analyte, or the second protein-binding moiety. The cross-linking may be indirect, for example, exposing the complex to antibodies against two or more of the first protein-binding moiety, the analyte, or the second protein-binding moiety. The type of cross-linking process used will depend on the cross-linking agent selected. A variety of suitable cross-linking agents and associated processes are available, non-limiting examples of which are provided herein, such was with respect to complexes and compositions disclosed herein.

### EXAMPLES

### Example 1: Multiplexed Sandwich Immunoassays

Methods described herein identify both the capture and detection antibodies in immunoassays such as multiplexed sandwich immunoassays.

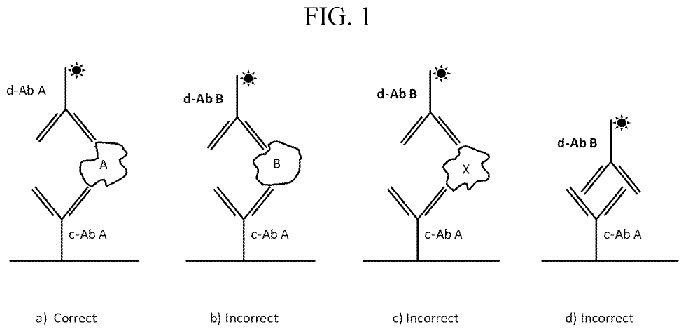

In a typical multiplexed sandwich immunoassay, a false signal can be produced when an incorrect detection antibody binds to a bead or an array element that is coated with a capture antibody. FIG. 1A illustrates a correct or specific signal, and FIGS. 1B-D illustrate examples of incorrect signal. The type of incorrect interaction illustrated in FIG. 1C is particularly problematic when measuring the concentration of analytes in real biological samples, such as serum or plasma. In such samples, there is a great variety of components (denoted “X” in the figure) that could bridge a wrong detection antibody to a capture antibody, and lead to an incorrect result. While interactions as in FIG. 1B and FIG. 1D can be evaluated in an artificial matrix, the interaction as in FIG. 1C is specific to individual samples, and thus is a particularly insidious type of false signal.

Currently available commercial multiplexed immunoassays detect the presence of detection antibodies on specific capture beads or array spots. However, they do not identify which detection antibody is present on any given capture surface, and instead, rely on the assumed specificity of the antibodies.

While carefully selected antibodies have a high degree of specificity, they are not infinitely specific. In fact, most antibodies will bind certain non-target proteins with low affinity. In practice, this limits the degree of multiplexing that can be achieved while avoiding non-specific interactions. This is one reason why most currently available commercial multiplexed assay panels are limited to 10-20 analytes, or fewer.

To address the need for improved multiplex protein detection methods, provided herein are immunoassays where the identity of both the capture and detection antibody is recognized. This enables a much higher degree of specificity than previously achievable when measuring analytes simultaneously. The approach also offers an almost unlimited degree of multiplexing through the readout of polynucleotide barcodes (e.g., DNA barcodes).

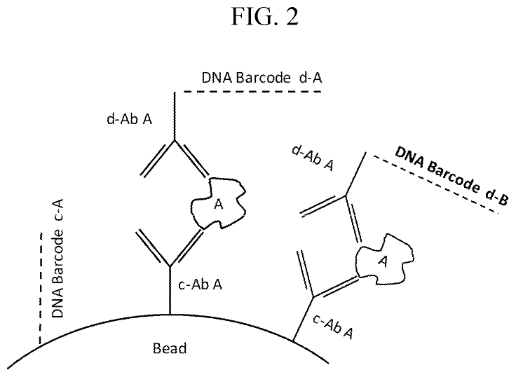

FIG. 2 shows an example of a bead coated with capture antibodies specific to analyte A (c-Ab A). The bead also carries a DNA barcode that identifies it as capture Bead A (DNA barcode c-A). The detection antibodies carry their own unique DNA barcodes. The illustration shows both a specific binding reaction (c-Ab A with d-Ab A), and a non-specific binding reaction (c-Ab A with d-Ab B).

Once the sandwich immunoassay reaction is carried out, the beads are read out in two sequencing reactions. First, a primer is introduced that allows all the detection barcodes to be read out by SBS (sequencing by synthesis). With four possible bases at each position, only a modest number of sequencing cycles is required. For example, in just five bases, 4\hat{ }5=1024 combinations can be encoded. Ten bases allow for 4¹⁰, or −1 million unique barcodes. Barcodes can be chosen to be as different as possible in the large space of available combinations.

In each sequencing cycle, the signal in four (4) fluorescent channels is read out, corresponding to each of the four bases. Once the desired number of sequencing cycles are complete, the linear decomposition of the signal into the known barcode sequences will produce the amplitude (or amount) of each barcoded antibody that was present. Thus, one can discriminate between the signals generated by the specific detection antibodies vs. the non-specific antibodies.

After the barcodes on the detection antibodies are read out, the extension can be permanently terminated, or the primers can be removed, or the barcode sequence can be chosen so that the extension is physically limited (i.e. reaches the end of the template). Then, a different primer can be introduced to read out the barcodes on the beads. These barcodes can either be attached directly to the beads, or attached to the capture antibodies. Since all that is required is reading out the identity of the bead, and there is only a single bar code sequence per bead (in multiple copies), this readout can be very short—e.g. five cycles to read out up to 1024 unique beads.

### Example 2: Bead-Well Format

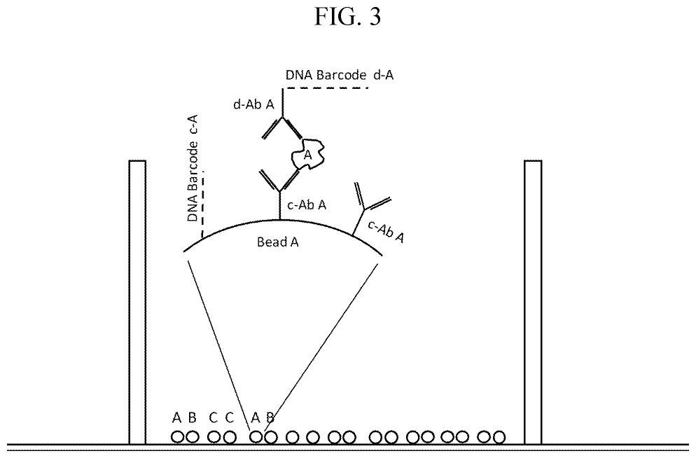

Methods as described herein may be carried out in a number of different formats including beads in a well plate. FIG. 3 shows a set of beads at the bottom of a well plate. Commonly used well plates have 96 or 384 wells. Other well-plate formats, such as 24, 48, 1536, and 9600 wells, etc. could also be used.

A standard 96-well plate would have a well diameter of ˜6 mm. If 1-micron diameter beads are used as the capture beads, the surface could accommodate ˜30 million beads in a monolayer. It may be preferable to use fewer beads to distinctly image individual beads, and thus would be sparsely arranged on the surface. Another approach is to pattern the bottom surface of the well to form a regular array of beads. This would allow a regularly spaced, high-density arrangement of beads, thus maximizing the number of beads that can be distinctly imaged at the desired inter-bead spacing.

Only a moderate number of beads would be sufficient for accurate measurement of the concentration of the target protein.

Even with larger beads and smaller wells, it would be possible to measure a large number of analytes simultaneously. For example, in a 3 mm diameter well (approximate size for 384-well plates), with 3 μm diameter beads, there would be room for ˜1 million beads; even a sparse spacing of only 10% coverage would have room for 100,000 beads. With 100 beads representing each analyte, this would allow for 1,000 analytes to be measured simultaneously. In embodiments, about or at least about 100, 500, 1000, 2500, 5000, or more analytes are assayed simultaneously. In embodiments, 100-5000, 250-2500, or 500-1000 analytes are assayed simultaneously. Assaying may include determining the presence, absence, or amount (absolute or relative) of an analyte in a sample.

### Example 3: Protein-Protein Interactions

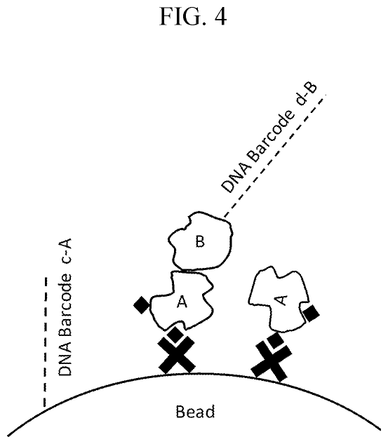

In addition to multiplexed immunoassays, the proposed approach can be used to measure protein-protein interactions. As depicted in FIG. 4, protein-protein interactions can be measured with DNA barcodes. There are beads coated with specific proteins, and another set of proteins labeled with DNA barcodes. Protein A is coated onto a capture bead, for example by using biotinylated proteins on a streptavidin-coated bead. The bead also carries a DNA barcode (capture-A). Similarly, other beads are coated with other proteins and corresponding barcodes. The binding of proteins in solution is detected by reading their associated barcode (shown as Protein B with DNA barcode d-B).

In this system, for example, 100 distinct bead types could be coated with capture proteins, and the same set or a different set of proteins would be labeled with barcodes. On each bead, the amount of each of the labeled (barcoded) proteins would be measured simultaneously, by the same sequencing readout as described in Example 1 for multiplexed immunoassays. After the detection barcodes are read out, a different primer would be used to initiate a readout of the capture barcodes. In one well, a set of 100×100 protein interactions could readily be measured. Even as many as 1000×1000 interactions could potentially be measured in a single well. In embodiments, about or at least about 1000, 10000, 50000, 100000, 500000, 1000000, or more interactions are assayed simultaneously. In embodiments, 1000-1000000, 10000-500000, or 50000-100000 interactions are assayed simultaneously.

### Example 4: Multiplexed Serology

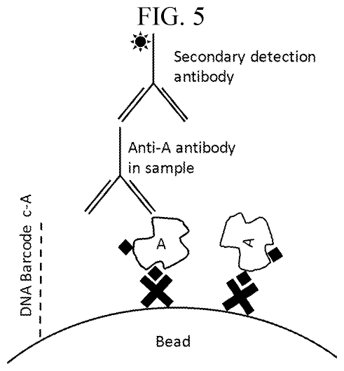

Another application of direct sequencing-based readout on beads is multiplexed serology. FIG. 5 shows beads coated with specific proteins, or other antigens. For example, all of the proteins encoded by the 20,000 genes in the human genome could be coated on individual beads. Antibodies present in blood are screened for recognition/binding to these proteins (auto-antigens). Similarly, samples are screened for antibodies specific to viral, bacterial or other microbial antigens.

In this example, only the barcodes on the beads are read out. The antibodies in the sample are measured via a secondary detection antibody with a fluorescent label, for example. A further extension of the multiplexed serology approach is to subtype the antibodies using subtype-specific secondary antibodies; these could be labeled by unique DNA barcodes, and read out the same way as described above.

### Numbered Embodiments

P1. A complex comprising:

a substrate;

a first protein-binding moiety attached to the substrate;

a substrate polynucleotide barcode attached to the substrate or to the first protein-binding moiety;

a second protein-binding moiety, wherein (i) the first protein-binding moiety and the second protein-binding moiety are proteins bound to each other, or (ii) the first protein-binding moiety and the second protein-binding moiety are bound to an analyte;

a protein polynucleotide barcode attached to the second protein-binding moiety; and

a complementary strand hybridized to one or more of the substrate polynucleotide barcode, or the protein polynucleotide barcode, wherein the complementary strand comprises a labeled nucleotide.

P2. The complex of embodiment P1, wherein (i) the first protein-binding moiety is a first protein or an aptamer, and/or (ii) the second protein-binding moiety is a second protein or an aptamer.
P3. The complex of embodiment P2, wherein the first protein-binding moiety is an antibody, an antigen-binding antibody fragment, an antigen, a receptor, or a ligand.
P4. The complex of embodiment P3, wherein the first protein-binding moiety is an antibody or an antigen-binding fragment thereof.
P5. The complex of any one of embodiments P2-P4, wherein the second protein-binding moiety is an antibody, an antigen-binding antibody fragment, an antigen, a receptor, or a ligand.
P6. The complex of embodiment P5, wherein the second protein-binding moiety is an antibody or an antigen-binding fragment thereof.
P7. The complex of any one of embodiments P1-P6, wherein the second protein-binding moiety and the first protein-binding moiety are non-covalently bound to an analyte.
P8. The complex of embodiment P7, wherein the analyte is a protein, an antibody, an antigen, or a ligand.
P9. The complex of any one of embodiments P1-P8, wherein the substrate is a bead.
P10. The complex of any one of embodiments P1-P9, wherein the substrate polynucleotide barcode, the protein polynucleotide barcode, or both are 3-15 nucleotides in length.
P11. The complex of any one of embodiments P1-P10, wherein (i) the substrate polynucleotide barcode forms a portion of a polynucleotide that is 10-50 nucleotides in length or 20-30 nucleotides in length; and/or (ii) the protein polynucleotide barcode forms a portion of a polynucleotide that is 10-50 nucleotides in length or 20-30 nucleotides in length.
P12. The complex of any one of embodiments P1-P11, wherein the first protein-binding moiety and the second protein-binding moiety are crosslinked.
P13. The complex of any one of embodiments P1-P12, wherein the complementary strand is formed by (i) extension of a polynucleotide primer, or (ii) extension of a 3′-end of a polynucleotide comprising the polynucleotide barcode to which the complementary strand is hybridized.
P14. A composition comprising a complex of any one of embodiments P1-P13, and further comprising a plurality of substrates and a plurality of different second protein-binding moieties, wherein (a) each substrate of the plurality of substrates is attached to a different first protein-binding moiety, (b) each substrate or each different first protein-binding moiety is attached to a different substrate polynucleotide barcode that uniquely identifies each of the different first protein-binding moieties, and (c) each different second protein-binding moiety of the plurality of different second protein-binding moieties is attached to a different protein polynucleotide barcode that uniquely identifies each of the plurality of different second protein-binding moieties.
P15. The composition of embodiment P14, wherein the plurality of substrates comprises at least 10, 100, 1000, 10000, 20000, 50000, or 100000 substrates.
P16. The composition of embodiment P14 or P15, wherein the plurality of different second proteins comprises at least 10, 100, 1000, 10000, 20000, 50000, or 100000 different second protein-binding moieties.
P17. The composition of any one of embodiments P14-P16, wherein each of the plurality of substrates is a bead.
P18. The composition of any one of embodiments P14-P17, wherein the plurality of substrates are in a container.
P19. The composition of any one of embodiments P14-P18, wherein the container is a well of a multi-well plate.
P20. The composition of any one of embodiments P14-P19, wherein none of the substrate polynucleotide barcodes is the same as any of the protein polynucleotide barcodes.
P21. A method of identifying a protein interaction, the method comprising:

(a) contacting a plurality of substrates with a plurality of different second protein-binding moieties, wherein (i) each substrate of the plurality of substrates is attached to a different first protein-binding moiety, (ii) each of the different first protein-binding moieties is a different first protein, (iii) each substrate or each different first protein-binding moiety is attached to a different substrate polynucleotide barcode, (iv) each substrate polynucleotide barcode uniquely identifies each of the different first protein-binding moieties, (v) each of the plurality of different second protein binding-moieties is a different second protein attached to a label, thereby forming a complex comprising one of the substrates attached to one of the different first proteins, one of the substrate polynucleotide barcodes, and one of the different second proteins attached to the label; and

(b) synthesizing and detecting a complementary strand of the substrate polynucleotide barcode within the complex, thereby identifying an interaction between the different first protein and the different second protein within the complex.

P22. A method of detecting an analyte in a sample, the method comprising:

(a) contacting a plurality of substrates with the sample and a plurality of different second protein-binding moieties, wherein (i) each substrate of the plurality of substrates is attached to a different first protein-binding moiety, (ii) each substrate or each different first protein-binding moiety is attached to a different substrate polynucleotide barcode, (iii) each substrate polynucleotide barcode uniquely identifies each of the different first protein-binding moieties, and (iv) each of the plurality of different second protein-binding moieties is attached to a label, thereby forming a complex comprising one of the substrates attached to one of the different first protein-binding moieties, one of the substrate polynucleotide barcodes, the analyte, and one of the second protein-binding moieties attached to the label;

(b) synthesizing and detecting a complementary strand of the substrate polynucleotide barcode within the complex, thereby detecting the analyte.

P23. The method of embodiment P22, wherein contacting with the sample and contacting with the plurality of different second protein-binding moieties is performed sequentially.
P24. The method of embodiment P22 or P23, wherein detecting the analyte comprises measuring a level of the analyte in the sample.
P25. The method of embodiment P24, wherein measuring the level of the analyte in the sample comprises forming a plurality of the complexes, and quantifying the labels that are present in the complexes.
P26. The method of any one of embodiments P22-P25, wherein the analyte is a protein, an antibody, an antigen, or a ligand.
P27. The method of embodiment P26, wherein the analyte is a human protein, a bacterial protein, or a viral protein.
P28. The method of any one of embodiments P22-P27, further comprising diagnosing a condition of a subject based on detecting the analyte.
P29. The method of any one of embodiments P22-P28, wherein (i) the first protein-binding moiety is a first protein or an aptamer, and/or (ii) the second protein-binding moiety is a second protein or an aptamer.
P30. The method of any one of embodiments P21-P29, wherein (i) the label comprises a protein polynucleotide barcode, (ii) each of the plurality of different second protein-binding moieties is attached to a different protein polynucleotide barcode that uniquely identifies each of the plurality of different second protein-binding moieties, and (iii) the method further comprises synthesizing and detecting a complementary strand of the protein polynucleotide barcode within the complex.
P31. The method of any one of embodiments P21-P30, wherein synthesizing and detecting the complementary strand comprises polymerizing the complementary strand from labeled nucleotides, and determining the sequence of the complementary strand from the sequence of the labels of the labeled nucleotides.
P32. The method of embodiment P31, wherein polymerizing the complementary strand comprises (i) extension of a polynucleotide primer, or (ii) extension of a 3′-end of a polynucleotide comprising the polynucleotide barcode to which the complementary strand is hybridized.
P33. The method of any one of embodiments P21-P32, wherein the plurality of different first protein-binding moieties comprise one or more of an antibody, an antigen-binding antibody fragment, an antigen, a receptor, or a ligand.
P34. The method of embodiment P33, wherein the plurality of different first protein-binding moieties comprise antibodies or antigen-binding fragments thereof.
P35. The method of any one of embodiments P21-P34, wherein the plurality of different first protein-binding moieties comprises a plurality of human proteins, a plurality of bacterial proteins, a plurality of viral proteins, a plurality of proteins having a mutation associated with a disease or condition, or a combination thereof.
P36. The method of any one of embodiments P21-P35, wherein the plurality of different second protein-binding moieties comprise one or more of an antibody, an antigen-binding antibody fragment, an antigen, a receptor, or a ligand.
P37. The method of embodiment P36, wherein the plurality of different second protein-binding moieties comprise antibodies or antigen-binding fragments thereof.
P38. The method of any one of embodiments P21-P37, wherein the substrate polynucleotide barcode, the protein polynucleotide barcode, or both are 3-15 nucleotides in length.
P39. The method of any one of embodiments P21-P38, wherein (i) the substrate polynucleotide barcode forms a portion of a polynucleotide that is 10-50 nucleotides in length or 20-30 nucleotides in length; and/or (ii) the protein polynucleotide barcode forms a portion of a polynucleotide that is 10-50 nucleotides in length or 20-30 nucleotides in length
P40. The method of any one of embodiments P21-P39, wherein the substrate polynucleotide barcodes collectively, the protein polynucleotide barcodes collectively, or both are defined by a nucleotide sequence comprising one or more degenerative or semi-degenerative sequences.
P41. The method of any one of embodiments P21-P40, wherein each of the plurality of substrates is a bead.
P42. The method of any one of embodiments P21-P41, wherein each substrate is separated from the plurality of substrates prior to synthesizing or detecting the complementary strands.
P43. The method of embodiment P42, wherein separating the substrates comprises arranging the substrates on an array or in an emulsion.
P44. The method of any one of embodiments P21-P43, wherein the plurality of substrates comprises at least 10, 100, 1000, 10000, 20000, 50000, or 100000 substrates, each of which is attached to a different first protein-binding moiety.
P45. The method of any one of embodiments P21-P44, wherein the plurality of different second proteins comprises at least 10, 100, 1000, 10000, 20000, 50000, or 100000 different second protein-binding moieties.
P46. The method of any one of embodiments P21-P45, wherein the plurality of substrates are in a container.
P47. The method of embodiment P46, wherein the container is a well of a multi-well plate.
P48. The method of any one of embodiments P30-P47, wherein none of the substrate polynucleotide barcodes is the same as any of the protein polynucleotide barcodes.
P49. The method of any one of embodiments P30-P48, wherein the complementary strand of the substrate polynucleotide barcode within the complex is synthesized before or after the complementary strand of the protein polynucleotide barcode within the complex.
P50. The method of any one of embodiments P21-P49, further comprising crosslinking the first protein-binding moiety with the second protein-binding moiety.
P51. The method of embodiment P50, wherein the crosslinking comprises forming covalent bonds between two or more of the first protein-binding moiety, the analyte, or the second protein-binding moiety.
P52. The method of embodiment P50, wherein the crosslinking comprises exposing the complex to antibodies against two or more of the first protein-binding moiety, the analyte, or the second protein-binding moiety.

1. A complex comprising:
a substrate;
a first protein-binding moiety attached to the substrate;
a substrate polynucleotide barcode attached to the substrate or to the first protein-binding moiety;
a second protein-binding moiety, wherein (i) the first protein-binding moiety and the second protein-binding moiety are proteins bound to each other, or (ii) the first protein-binding moiety and the second protein-binding moiety are bound to an analyte;
a protein polynucleotide barcode attached to the second protein-binding moiety; and
a complementary strand hybridized to one or more of the substrate polynucleotide barcode, or the protein polynucleotide barcode, wherein the complementary strand comprises a labeled nucleotide.

2. The complex of claim 1, wherein (i) the first protein-binding moiety is a first protein or an aptamer, an antibody, an antigen-binding antibody fragment, an antigen, a receptor, or a ligand, and/or (ii) the second protein-binding moiety is a second protein or an aptamer, an antibody, an antigen-binding antibody fragment, an antigen, a receptor, or a ligand.

3.-6. (canceled)

7. The complex of claim 1, wherein the second protein-binding moiety and the first protein-binding moiety are non-covalently bound to an analyte, wherein the analyte is a protein, and antibody, an antigen, or a ligand.

8. (canceled)

9. The complex of claim 1, wherein the substrate is a bead.

10. The complex of claim 1, wherein the substrate polynucleotide barcode, the protein polynucleotide barcode, or both are 3-15 nucleotides in length.

11. The complex of claim 1, wherein (i) the substrate polynucleotide barcode forms a portion of a polynucleotide that is 10-50 nucleotides in length or 20-30 nucleotides in length; and/or (ii) the protein polynucleotide barcode forms a portion of a polynucleotide that is 10-50 nucleotides in length or 20-30 nucleotides in length.

12. The complex of claim 1, wherein the first protein-binding moiety and the second protein-binding moiety are crosslinked.

13. The complex of claim 1, wherein the complementary strand is formed by (i) extension of a polynucleotide primer, or (ii) extension of a 3′-end of a polynucleotide comprising the polynucleotide barcode to which the complementary strand is hybridized.

14. A composition comprising a complex of claim 1, and further comprising a plurality of substrates and a plurality of different second protein-binding moieties, wherein (a) each substrate of the plurality of substrates is attached to a different first protein-binding moiety, (b) each substrate or each different first protein-binding moiety is attached to a different substrate polynucleotide barcode that uniquely identifies each of the different first protein-binding moieties, and (c) each different second protein-binding moiety of the plurality of different second protein-binding moieties is attached to a different protein polynucleotide barcode that uniquely identifies each of the plurality of different second protein-binding moieties.

15. The composition of claim 14, wherein the plurality of substrates comprises at least 10, 100, 1000, 10000, 20000, 50000, or 100000 substrates.

16.-20. (canceled)

21. A method of identifying a protein interaction, the method comprising:
(a) contacting a plurality of substrates with a plurality of different second protein-binding moieties, wherein (i) each substrate of the plurality of substrates is attached to a different first protein-binding moiety, (ii) each of the different first protein-binding moieties is a different first protein, (iii) each substrate or each different first protein-binding moiety is attached to a different substrate polynucleotide barcode, (iv) each substrate polynucleotide barcode uniquely identifies each of the different first protein-binding moieties, (v) each of the plurality of different second protein binding-moieties is a different second protein attached to a label, thereby forming a complex comprising one of the substrates attached to one of the different first proteins, one of the substrate polynucleotide barcodes, and one of the different second proteins attached to the label; and
(b) synthesizing and detecting a complementary strand of the substrate polynucleotide barcode within the complex, thereby identifying an interaction between the different first protein and the different second protein within the complex.

22. A method of detecting an analyte in a sample, the method comprising:
(a) contacting a plurality of substrates with the sample and a plurality of different second protein-binding moieties, wherein (i) each substrate of the plurality of substrates is attached to a different first protein-binding moiety, (ii) each substrate or each different first protein-binding moiety is attached to a different substrate polynucleotide barcode, (iii) each substrate polynucleotide barcode uniquely identifies each of the different first protein-binding moieties, and (iv) each of the plurality of different second protein-binding moieties is attached to a label, thereby forming a complex comprising one of the substrates attached to one of the different first protein-binding moieties, one of the substrate polynucleotide barcodes, the analyte, and one of the second protein-binding moieties attached to the label;
(b) synthesizing and detecting a complementary strand of the substrate polynucleotide barcode within the complex, thereby detecting the analyte.

23. The method of claim 22, wherein contacting with the sample and contacting with the plurality of different second protein-binding moieties is performed sequentially.

24. The method of claim 22, wherein detecting the analyte comprises measuring a level of the analyte in the sample.

25. (canceled)

26. The method of claim 22, wherein (i) the first protein-binding moiety is a first protein or an aptamer, and/or (ii) the second protein-binding moiety is a second protein or an aptamer, and wherein the analyte is a protein, an antibody, an antigen, or a ligand.

27. (canceled)

28. The method of claim 22, further comprising diagnosing a condition of a subject based on detecting the analyte.

29. (canceled)

30. The method of claim 21, wherein (i) the label comprises a protein polynucleotide barcode, (ii) each of the plurality of different second protein-binding moieties is attached to a different protein polynucleotide barcode that uniquely identifies each of the plurality of different second protein-binding moieties, and (iii) the method further comprises synthesizing and detecting a complementary strand of the protein polynucleotide barcode within the complex.

31. The method of claim 21, wherein synthesizing and detecting the complementary strand comprises polymerizing the complementary strand from labeled nucleotides, and determining the sequence of the complementary strand from the sequence of the labels of the labeled nucleotides, wherein polymerizing the complementary strand comprises (i) extension of a polynucleotide primer, or (ii) extension of a 3′-end of a polynucleotide comprising the polynucleotide barcode to which the complementary strand is hybridized.

32. (canceled)

33. The method of claim 21, wherein (i) the plurality of different first protein-binding moieties comprise one or more of an antibody, an antigen-binding antibody fragment, an antigen, a receptor, or a ligand, (ii) the plurality of different second protein-binding moieties comprise one or more of an antibody, an antigen-binding antibody fragment, an antigen, a receptor, or a ligand, and/or (iii) the plurality of different first protein-binding moieties comprises a plurality of human proteins, a plurality of bacterial proteins, a plurality of viral proteins, a plurality of proteins having a mutation associated with a disease or condition, or a combination thereof.

34.-37. (canceled)

38. The method of claim 21, wherein the substrate polynucleotide barcode, the protein polynucleotide barcode, or both are 3-15 nucleotides in length.

39.-49. (canceled)

50. The method of claim 21, further comprising crosslinking the first protein-binding moiety with the second protein-binding moiety, wherein (i) the crosslinking comprises forming covalent bonds between two or more of the first protein-binding moiety, the analyte, or the second protein-binding moiety, and/or (ii) the crosslinking comprises exposing the complex to antibodies against two or more of the first protein-binding moiety, the analyte, or the second protein-binding moiety.

51. (canceled)

52. (canceled)
